(19) United States
(12) Patent Application Publication
(10) Pub. No.: US 2012/0214965 A1
(43) Pub. Date: Aug. 23, 2012

(54) GLARGINE PROINSULIN AND METHODS OF PRODUCING GLARGINE INSULIN ANALOGS THEREFROM

(75) Inventors: Ronald E. Zimmerman, Greenwood, IN (US); David John Stokell, Indianapolis, IN (US); Michael Patrick Akers, Indianapolis, IN (US)

(73) Assignee: Elona Biotechnologies, Greenwood, IN (US)

(21) Appl. No.: 13/032,806

(22) Filed: Feb. 23, 2011

(51) Int. Cl. C07K 14/62; C12N 1/21; C12P 21/02; C12N 15/63

(52) U.S. Cl. 530/303; 435/320.1; 435/252.33; 435/69.4

(57) ABSTRACT

Glargine proinsulin sconstructs that have a modified C-peptide amino acid and/or nucleic acid sequence for producing glargine insulin analogs are provided. Highly efficient processes for preparing the glargine insulin analogs and improved preparations containing the glargine insulin analogs prepared according to the methods described herein are also provided.

### SEQUENCE LISTING

The instant application contains a Sequence Listing which has been submitted in ASCII format via EFS-Web and is hereby incorporated by reference in its entirety. Said ASCII copy, created on Feb. 21, 2011, is named 34344516.txt and is 21,956 bytes in size.

### FIELD OF THE INVENTION

The invention relates to compositions and preparations that comprise glargine proinsulin, in particular glargine proinsulin with modified C-peptide sequences. The invention also relates to methods of manufacture for manufacturing glargine insulin analogs from modified proinsulin sequences.

### BACKGROUND

Insulin is a hormone that regulates glucose metabolism in animals. Insulin is a polypeptide hormone secreted by beta-cells of the pancreas. This hormone is made up of two polypeptide chains, an A-chain of 21 amino acids, and a B-chain of 30 amino acids. These two chains are linked to one another in the mature form of the hormone by two interchain disulphide bridges. The A-chain also features one intra-chain disulphide bridge.

Insulin analogs are altered forms of native insulin that are available to the body for performing the same action as native insulin. A specific insulin analog known as glargine insulin has also been described in U.S. Pat. Nos. 5,547,930, 5,618,913, and 5,834,422. This analog is used in the treatment of diabetes. Glargine insulin is characterized as a slow release insulin analog that controls blood sugar when no food is being digested. Glargine insulin may form a hexamer when injected subcutaneously into the patient. This insulin analog has been available commercially as LANTUS® (Sanofi Aventis). LANTUS® is an insulin analog wherein the molecule includes a Gly(A₂₁)-Arg(B₃₁)-Arg(B₃₂) amino acid sequence.

Native insulin is a hormone that is synthesized in the body in the form of a single-chain precursor molecule, proinsulin. Proinsulin is a molecule comprised of a prepeptide of 24 amino acids, followed by the B-chain peptide, a C-peptide of 35 amino acids, and the A-chain peptide. The C-peptide of this precursor insulin molecule (“proinsulin”) contains the two amino acids, lysine-arginine (KR) at its carboxy end (where it attaches to the A-chain), and the two amino acids, arginine-arginine (RR) at its amino end (where it attaches to the B-chain). In the mature insulin molecule, the C-peptide is cleaved away from the peptide so as to leave the A-chain and the B-chain connected directly to one another in its active form.

Molecular biology techniques have been used to produce human proinsulin. In this regard, three major methods have been used for the production of this molecule. Two of these methods involve Escherichia coli, with either the expression of a large fusion protein in the cytoplasm (Chance et al. (1981), and Frank et al. (1981) in Peptides: Proceedings of the 7^{th} American Peptide Chemistry Symposium (Rich, D. and Gross, E., eds.), pp. 721-728, 729-739, respectively, Pierce Chemical Company, Rockford, Ill.), or the use of a signal peptide to enable secretion into the periplasmic space (Chan et al. (1981) P.N.A.S., USA., 78:5401-5404). A third method utilizes yeast, especially Saccharomyces cerevisiae, to secrete the insulin precursor into the medium (Thim, et al. (1986), P.N.A.S., USA., 83: 6766-6770).

Chance et al. reported a process for preparing insulin by producing each of the A and B chains of insulin in the form of a fusion protein by culturing E. coli that carries a vector compromising a DNA encoding the fusion protein, cleaving the fusion protein with cyanogen bromide to obtain the A and the B chains, sulfonating the A and B chains to obtain sulfonated chains, reacting the sulfonated B chain with an excess amount of the sulfonated A chain; and then purifying the resultant products to obtain insulin. Drawbacks associated with this process are that it requires two fermentation processes and the requirement of a reaction step for preparing the sulfonated A chain and the sulfonated B chain. This results in a low insulin yield.

Frank et al. described a process for preparing insulin in the form of a fusion protein in E. coli. In this process, proinsulin is produced in the form of a fusion protein by culturing E. coli which carries a vector comprising a nucleic acid sequence (DNA) encoding for the fusion protein, cutting the fusion protein with cyanogen bromide to obtain proinsulin, sulfonating the proinsulin and separating the sulfonated proinsulin, refolding the sulfonated proinsulin to form correct disulfide bonds, treating the refolded proinsulin with trypsin and carboxypeptidase B, and then purifying the resultant product to obtain insulin. However, the yield of the refolded proinsulin having correctly folded disulfide bonds is reported to sharply decrease as the concentration of the proinsulin increases. This is allegedly due to, at least among other reasons, misfolding of the protein, and some degree of polymerization being involved. Hence, the process entails the inconvenience of using laborious purification steps during the recovery of proinsulin.

Thim et al. reported a process for producing insulin in yeast, Saccharomyces cerevisiae. This process has the steps of producing a single chain insulin analog having a certain amino acid sequence by culturing Saccharomyces cerevisiae cells, and isolating insulin therefrom through the steps of: purification, enzyme reaction, acid hydrolysis and a second purification. This process, however, results in an unacceptably low yield of insulin.

The role of the native C-peptide in the folding of proinsulin is not precisely known. The dibasic terminal amino acid sequence at both ends of the C-peptide sequence has been considered necessary to preserve the proper processing and/or folding of the proinsulin molecule to insulin.

Other amino acids within the C-peptide sequence, however, have been modified with some success. For example, Chang et al. (1998) (Biochem. J., 329:631-635) described a shortened C-peptide of a five (5) amino acid length, -YPGDV- (SEQ ID NO: 1), that includes a preserved terminal di-basic amino acid sequence, RR at one terminal end, and KR at the other terminal end, of the peptide. Preservation of the dibasic amino acid residues at the B-chain-C peptide and C-peptide-A-chain junctures is taught as being a minimal requirement for retaining the capacity for converting the proinsulin molecule into a properly folded mature insulin protein. The production of the recombinant human insulin was described using E. coli with a shortened C-peptide having a dibasic amino acid terminal sequence. U.S. Pat. No. 5,962,267 also describes dibasic terminal amino acid sequences at both ends of the C-peptide.

One of the difficulties and/or inefficiencies associated with the production of recombinant insulin employing a proinsulin construct having the conserved, terminal di-basic amino acid sequence in the C-peptide region is the presence of impurities, such as Arg(A₀)-insulin, in the reaction mixture, once enzymatic cleavage to remove the C-peptide is performed. This occurs as a result of misdirected cleavage of the proinsulin molecule so as to cleave the C-peptide sequence away from the A-chain at this juncture, by the action of trypsin. Trypsin is a typical serine protease, and hydrolyses a protein or peptide at the carboxyl terminal of an arginine or lysine residue (Enzymes, pp. 261-262 (1979), ed. Dixon, M. & Webb, E. C. Longman Group Ltd., London). This unwanted hydrolysis results in the unwanted Arg(A₋)-insulin by-product, and typically constitutes about 10% of the reaction yield. Hence, an additional purification step is required. The necessity of an additional purification step makes the process much more time consuming, and thus expensive, to use. Moreover, an additional loss of yield may be expected from the necessity of this additional purification step.

Others have described the use of proinsulin constructs that do not have a conserved terminal dibasic amino acid sequence of the C-peptide region. For example, U.S. Pat. No. 6,777,207 (Kjeldsen et al.) relates to a novel proinsulin peptide construct containing a shortened C-peptide that includes the two terminal amino acids, glycine-arginine or glycine-lysine at the carboxyl terminal end that connects to the A-chain of the peptide. The B-chain of the proinsulin construct described therein has a length of 29 amino acids, in contrast to the native 30 amino acid length of the native B-chain in human insulin. The potential effects of this change to the native amino acid sequence of the B-chain in the human insulin produced are yet unknown. Methods of producing insulin using these proinsulin constructs in yeast are also described. Inefficiencies associated with correct folding of the mature insulin molecule when yeast is used as the expression host, render this process, among other things, inefficient and more expensive and time consuming to use. In addition, yeast provides a relatively low insulin yield, due to the intrinsically low expression levels of a yeast system as compared to E. coli.

As evidenced from the above review, a present need exists for a more efficient process for production of glargine insulin analogs that is efficient and that may also improve and/or preserve acceptable production yield requirements of the pharmaceutical industry.

The above references are incorporated by reference herein where appropriate for appropriate teachings of additional or alternative details, features and/or technical background.

### SUMMARY OF THE INVENTION

The present invention provides processes for using a modified proinsulin sequence to produce glargine insulin analogs. The modified proinsulin sequence has the formula

R₁—(B₁-B₂₉)—B₃₀—R₂—R₃—X—R₄—R₅-(A₁-A₂₀)-A₂₁-R₆ Formula I

wherein

R₁ is a tag sequence containing one or more amino acids, preferably with a C-terminal Arg or Lys, or R₁ is absent, with an Arg or Lys present prior to the start of the B chain;

(B₁-B₂₉) and (A₁-A₂₀) comprise amino acid sequences of native human insulin;

B₃₀ is Gly, Ala, Ser, Thr, Val, Leu, Ile, Asn, Gln, Cys, Met, Tyr, Phe, Pro, or Trp, preferably Thr;

R₂, R₃ and R₅ are Arg;

R₄ is any amino acid other than Gly, Lys or Arg or is absent, preferably Ala;

X is a sequence that comprises one or more amino acids or is absent, provided that X does not comprise a C-terminal Gly, Lys, or Arg when R₄ is absent;

A₂₁ is Gly, Ala, Val, Leu, Ile, Pro, Phe, Trp, Met, Ser, Thr, Tyr, Asp, or Glu, preferably Gly; and

R₆ is a tag sequence containing one or more amino acids, preferably with a N-terminal Arg or Lys, or R₆ is absent;

One aspect of the present invention is related to a process for producing glargine insulin analogs comprising the steps of culturing E. coli cells under conditions suitable for expression of a modified proinsulin sequence as provided in Formula I; disrupting the cultured E. coli cells to provide a composition comprising inclusion bodies containing the modified proinsulin sequence; solubilizing the composition of inclusion bodies; and recovering glargine insulin analogs from the solubilized composition.

Another aspect of the present invention is related to a process for producing glargine insulin analogs comprising the steps of: (a) providing a modified proinsulin sequence as provided for in Formula I; (b) folding the modified proinsulin sequence to provide a glargine proinsulin derivative peptide; (c) purifying the glargine proinsulin derivative using metal affinity chromatography; (d) enzymatically cleaving the glargine proinsulin derivative peptide to remove a connecting peptide and tag to provide an intermediate solution comprising glargine insulin analog; and (e) purifying the intermediate solution using chromatography columns to yield the glargine insulin analog.

Additional advantages, objects, and features of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE FIGURES

The invention will be described in detail with reference to the following drawings in which like reference numerals refer to like elements wherein.

FIG. 1, according to one aspect of the invention, is a vector map of plasmid pTrcHis2A (Kan) with a glargine proinsulin gene insert.

FIG. 2, according to one aspect of the invention, is a process flow scheme for the purification of glargine insulin analogs.

FIG. 3 is an analytical HPLC overlay of glargine product by the method (B) according to one aspect of the invention, compared with LANTUS® (A), manufactured by Sanofi Aventis.

### DETAILED DESCRIPTION

The present invention generally relates to the preparation of insulin analogs, specifically glargine insulin analog, from modified proinsulin sequences. Glargine insulin analog comprises a modified A-chain and B-chain having Gly(A₂₁), Arg(B₃₁), and Arg(B₃₂). Modified proinsulin sequences refer to a single-chain polypeptide that may be converted into human insulin or insulin analogs and comprise a connecting peptide (C-peptide) having at least one non-dibasic terminal amino acid sequence. In one embodiment, non-dibasic terminal amino acid sequences may comprise (any amino acid except Lys or Arg-Arg ((any except R or K)R), and more preferably (any amino acid except Gly, Lys, or Arg-Arg ((any except G, R, or K)R). In one embodiment the terminal amino acid sequence may comprise Ala-Arg. Advantageously, the positioning of these particular terminal amino acids in the C-peptide provides for an improved method for producing recombinant glargine insulin analog, having fewer steps, improved yields of the recombinant glargine insulin analog and less contaminating byproducts.

The process for producing glargine insulin analogs of the invention presents many advantages, among them the advantage of reducing and/or eliminating the presence of unwanted and contaminating cleavage by-products characteristic of conventional manufacturing processes for producing recombinant human insulin in E. coli. Previously undesirable by-products evident in yield mixtures using conventional methods of producing recombinant human insulin analogs included, by way of example, the production of an unwanted cleavage product, Arg(A₀)-insulin analogs. A highly efficient process for the production of recombinant human insulin analogs is presented that reduces and/or eliminates the presence of this and other unwanted and undesirable cleavage by-products, and that further presents the advantages of eliminating several time consuming, expensive, purification steps. A process having fewer technician-assisted steps is thus devised, and illustrates the additional advantage of eliminating the degree of inconsistency and/or error associated with technician assisted steps in the manufacturing process.

A second key advantage involves the citraconylation of Lys 29 of the B chain using citraconic anhydride. The lysine in the following sequence of pro-insulin; pro-lys-thr-arg-arg (SEQ ID NO: 24), is cleaved by trypsin during the transformation reaction at a rate of approximately 5-8%, which creates the desthreonine insulin contaminant. Citraconylation of the lysine prevents trypsin cleavage and in turn prevents desthreonine insulin. The citraconylation also decreases trypsin cleavage at the arginine at position B₃₁ of the C-peptide. Single arg(B₃₁)-insulin represents approximately 20-30% during the trypsin digest. However, when the lysine is blocked by citraconic anhydride, the level of arg(B₃₁)-insulin decreases to approximately 6-10% during the trypsin digest. Decreased levels of desthreonine insulin and arg(B₃₁)-insulin provides for a simpler purification, as both these impurities are difficult to remove due to their high similarity to final glargine material.

In one embodiment, the modified glargine proinsulin sequence of the present invention has the formula

R₁—(B₁-B₂₉)—B₃₀—R₂—R₃—X—R₄—R₅-(A₁-A₂₀)-A₂₁-R₆ Formula I

wherein

R₁ is a tag sequence containing one or more amino acids, preferably with a C-terminal Arg or Lys, or R₁ is absent with an Arg or Lys present prior to the start of the B chain;

(B₁-B₂₉) and (A₁-A₂₀) comprise amino acid sequences of native human insulin;

B₃₀ is Gly, Ala, Ser, Thr, Val, Leu, Ile, Asn, Gln, Cys, Met, Tyr, Phe, Pro, or Trp, preferably Thr;;

R₂, R₃ and R₅ are Arg;

R₄ is any amino acid other than Gly, Lys or Arg or is absent, preferably Ala;

X is a sequence comprising one or more amino acids or is absent, provided that X does not comprise a C-terminal Gly, Lys, or Arg when R₄ is absent;

A₂₁ is Gly, Ala, Val, Leu, Ile, Pro, Phe, Trp, Met, Ser, Thr, Tyr, Asp, or Glu, preferably Gly; and

R₆ is a tag sequence containing one or more amino acids, preferably with a N-terminal Arg or Lys, or R₆ is absent.

R₁ or R₆ in the modified glargine proinsulin of Formula I comprises a pre or post-peptide that may be a native pre-peptide or an N-terminal multiple His-tag sequence, or any other commercially available tag utilized for protein purification, e.g. DSBC, Sumo, Thioredein, T7, S tag, Flag Tag, HA tag, VS epitope, Pel B tag, Xpress epitope, GST, MBP, NusA, CBP, or GFP. In one embodiment at least one of R₁ or R₆ is present in Formula I. It is preferred that the terminal amino acid of the pre or post-peptide that connects to the B-chain or A-chain comprise Arg or Lys. Native pre-peptide has the sequence of MALWMRLLPLLALLALWGPDPAAA (SEQ ID NO: 2). In some embodiments, the N-terminal multiple His-tagged proinsulin construct comprises a 6-histidine (SEQ ID NO: 3) N-terminal tag and may have the sequence of MHHHHHHGGR (SEQ ID NO: 4). The modified proinsulin sequence may replace the native 24 amino acid pre-peptide with the 6-histidine (SEQ ID NO: 3) N-terminal tag sequence. In some embodiments, R₁ and/or R₆ may be a sequence of one or more amino acids, e.g., preferably from 1 to 30 and more preferably from 6 to 10.

Native insulin comprises an A-chain having the sequence GIVEQCCTSICSLYQLENYCN (SEQ ID NO: 5) and a B-chain having the sequence FVNQHLCGSHLVEALYLVCGERGFFYTPKT (SEQ ID NO: 6). According to the invention, the A-chain and B-chain of Formula I is modified from native insulin and contains at least one amino acid mutation, substitution, deletion, insertion, and/or addition. For glargine insulin analogs, preferably A₂₁ is modified and B₃₁ and B₃₂ of the B-chain are added. The asparagine (A₂₁) of native insulin is substituted with glycine and the two arginine amino acids are added to (B₃₀) of native insulin. In one embodiment, the two arginine amino acids may be amino acid residues from the C-peptide. In one embodiment, the A-chain and/or B-chain that is modified is a human insulin B-chain. In another embodiment, the A-chain and/or B-chain that is modified is porcine insulin B-chain.

As used in the description of the present invention, the term “connecting peptide” or “C-peptide” is meant the connecting moiety “C” of the B-C-A polypeptide sequence of a single chain proinsulin molecule. As in the native human proinsulin, the N-terminus of the C-peptide connects to C-terminus of the modified B-chain, e.g., position 30 of the B-chain, and the C-terminus of the C-peptide connects to N-terminus of the A-chain, e.g., position 1 of the A-chain.

In some embodiments, the C-peptide may have a sequence of Formula II:

R₂—R₃—X—R₄—R₅ Formula II

wherein R₂, R₃, R₄, R₅, and X have the same meaning as in Formula I. In one embodiment, X may be a sequence having up to 40 amino acids, preferably up to 35 amino acids or more preferably up to 30 amino acids. Although X may be any amino acid sequence, in one embodiment, X is preferably not EAEALQVGQVELGGGPGAGSLQPLALEGSLQ (SEQ ID NO: 7).

The C-peptide sequences of the present invention may include:

(SEQ ID NO: 8)
(1) RREAEDLQVGQVELGGGPGAGSLQPLALEGSLQAR;
(SEQ ID NO: 9)
(2) RREAEDLQVGQVGLGGGPGAGSLQPLALEGSLQAR;
(SEQ ID NO: 10)
(3) RREAEALQVGQVGLGGGPGAGSLQPLALEGSLQAR;
(SEQ ID NO: 11)
(4) RREAEDLQVGQVELGGGPGAGSLQPLAIEGSLQAR;
(SEQ ID NO: 12)
(5) RREAEDLQVGQVGLGGGPGAGSLQPLAIEGSLQAR;
(SEQ ID NO: 13)
(6) RREAEALQVGQVGLGGGPGAGSLQPLAIEGSLQAR;
or
(SEQ ID NO: 14)
(7) RREAEALQVGQVELGGGPGAGSLQPLALEGSLQAR.

Preferred modified glargine proinsulin sequences of the present invention may include:

(SEQ ID NO: 15)
(1)FVNQHLCGSHLVEALYLVCGERGFFYTPKTRREAEDLQVGQVELGGG
PGAGSLQPLALEGSLQARGIVEQCCTSICSLYQLENYCG;
(SEQ ID NO: 16)
(2)MHHHHHHGGRFVNQHLCGSHLVEALYLVCGERGFFYTPKTRREAEDL
QVGQVELGGGPGAGSLQPLALEGSLQARGIVEQCCTSICSLYQLENYCG;
(SEQ ID NO: 17)
(3)MALWMRLLPLLALLALWGPDPAAAFVNQHLCGSHLVEALYLVCGERG
FFYTPKTRREAEDLQVGQVELGGGPGAGSLQPLALEGSLQARGIVEQCCT
SICSLYQLENYCG;
(SEQ ID NO: 18)
(4)MHHHHHHGGRFVNQHLCGSHLVEALYLVCGERGFFYTPKTRREAEDL
QVGQVELGGGPGAGSLQPLALEGSLQARGIVEQCCTSICSLYQLENYCGR
HHHHHH;
(SEQ ID NO: 19)
(5)MHHHHHHGGRFVNQHLCGSHLVEALYLVCGERGFFYTPKTRREAEDL
QVGQVELGGGPGAGSLQPLALEGSLQARGIVEQCCTSICSLYQLENYCGK
HHHHHH;
(SEQ ID NO: 20)
(6)MRFVNQHLCGSHLVEALYLVCGERGFFYTPKTRREAEDLQVGQVELG
GGPGAGSLQPLALEGSLQARGIVEQCCTSICSLYQLENYCGRHHHHHH;
or
(SEQ ID NO: 21)
(7)MRFVNQHLCGSHLVEALYLVCGERGFFYTPKTRREAEDLQVGQVELG
GGPGAGSLQPLALEGSLQARGIVEQCCTSICSLYQLENYCGKHHHHHH.

The single chain glargine insulin analogs of the invention will include three (3) correctly positioned, disulphide bridges, as is characteristic of the native human insulin. In some embodiments, the folded modified proinsulin, or proinsulin derivative peptide, may include three (3) correctly positioned, disulphide bridges. In the production, the C-peptide of the glargine proinsulin derivative peptide is removed to produce the glargine insulin analog. Glargine insulin analogs of the invention have a sequence (SEQ ID NOS 22-23, respectively, in order of appearance) of Formula III, where the disulphide bridges are represented as —S—S—:

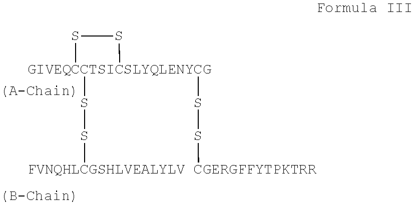

The present invention provides modified proinsulin sequences having the modified C-peptide and methods for using these in a process to provide high yields of mature recombinant glargine insulin analog. Advantageously, the positioning of these particular terminal amino acids in the C-peptide may provide for an improved method for producing recombinant glargine insulin analog, having fewer steps, improved yields of the recombinant glargine insulin analog and less contaminating byproducts.

As used in the description of the present invention, the terms “insulin precursor” or “proinsulin” are described as a single-chain polypeptide in which, by one or more subsequent chemical and/or enzymatic processes, may be converted into human insulin or insulin analog.

A proinsulin analog or modified proinsulin is defined as a proinsulin molecule having one or more mutations, substitutions, deletions, and or additions, of the A, B and/or C chains relative to the native human proinsulin nucleic acid sequence. The proinsulin analogs are preferably such wherein one or more of the naturally occurring nucleic acids have been substituted with another nucleic acid within a triplet encoding for a particular amino acid. For purposes of convenience, proinsulin analog is understood to refer to glargine proinsulin analog, unless otherwise specified.

The term “insulin analog” includes insulin molecules having one or more mutations, substitutions, deletions, additions, or modifications to one or more amino acids of a native insulin sequence. For example, in one embodiment, the native insulin sequence is porcine insulin, while in another embodiment, the native insulin sequence is human For purposes of convenience insulin analog is understood to refer to glargine insulin analog, unless otherwise specified.

The term “a” as used in the description of the present invention is intended to mean “one or more”, and is used to define both the singular and plural forms of the item or items to which it references, or to a feature or characteristic to which it refers. The use of the singular or plural in the claims or specification is not intended to be limiting in any way and also includes the alternative form.

The term “about” is intended to be inclusive of and to encompass both an exact amount as well as an approximate amount or range of values or levels of the item, ingredient, element, activity, or other feature or characteristic to which it references. Generally, and in some embodiments, the term “about” is intended to reference a range of values relatively close to the specific numerical value specifically identified. For example, “about 3 grams to about 5 grams” is intended to encompass a measure of in or around a value of 3 grams, concentration values between 3 grams and 5 grams, concentration values in and around 5 grams, as well as concentration values that are exactly 3 grams and exactly 5 grams.

As used in the description of the present process, a high protein concentration of the proinsulin or insulin analog product is defined as a protein yield concentration of at least about 3 grams/liter, or between about 3 grams to about 5 grams per liter. The expression yield to be expected may be defined as a protein/peptide yield that is sufficient to detect via polyacrylamide gel electrophoreses (PAGE).

The invention provides a process for producing highly purified glargine insulin analog that is more efficient than current techniques. The invention in a general and overall sense relates to an improved process for preparing a heterologous recombinant protein in a prokaryotic host cell. This process is characterized in that it employs a recombinant protein that provides a useful and efficiently processed modified proinsulin sequence analog as described herein.

The term “heterologous protein” is intended to mean that the protein in the prokaryotic host cell is not native, i.e., it occurs as a protein in peculiar or foreign (i.e., not native to) the host prokaryotic cell.

The term “recombinant” is intended to mean produced or modified by molecular-biological methods. For example, according to one embodiment, a recombinant protein is made using genetic engineering techniques and is not found in nature.

As used in the description of the present invention, the term “heterologous recombinant protein” is defined as any protein known to the skilled person in the molecular biological arts, such as, for example, insulin, insulin analog, proinsulin, proinsulin analog, C-peptide, and proteins containing these together with any other protein or peptide fragment.

Prokaryotic host cells may be any host cells known to the skilled artisan in the molecular biological arts, and by way of example, Escherichia coli. Such types of cells available from public collections and useful in the practice of the present invention include, by way of example, the Deutsche Sammlung von Mikrooganismen and Zellkulturen GmbH, raunschweig, Germany, e.g., E. coli Strain K12 JM107 (DSM 3950).

The following reference table, Table 1, provides the triplet codons corresponding to each of the various amino acids that are used in the description of the present invention. As will be understood by those of skill in the art, the amino acid that may be used in any particularly defined position as part of any of the peptide, protein, or constructs otherwise defined herein by reference to a particular nucleotide triplet base pair may be encoded by a number of different nucleotide triplets that function to encode the same amino acid. For example, where the amino acid of the sequence defined herein is alanine (Ala, or A), the triplet codon of nucleic acids that may encode for this amino acid are: GCT, GCC, GCA, or GCG. The following table illustrates this definition of variables at and substitutions as can be applied to all of the naturally occurring amino acids sequences of the disclosure.

TABLE 1

| | U | | | C | | | A | | | G | | | |
|---|---|---|---|---|---|---|---|---|---|---|---|---|---|
| U | UUU | | | UCU | | | UAU | | | UGU | | | U |
| | | {close oversize brace} | Phe | | | | | {close oversize brace} | Tyr | | | Cys | |
| | UUC | | | UCC | | | UAC | | | UGG | | | C |
| | UUA | | | UCA | {close oversize brace} | Ser | UAA | | Stop | UGA | | Stop | A |
| | | {close oversize brace} | Leu | | | | | | | | | | |
| | UUG | | | UCG | | | UAG | | Stop | UGG | | Trp | G |
| C | CUU | | | CCU | | | CAU | | | CGU | | | U |
| | | | | | | | | {close oversize brace} | His | | | | |
| | CUC | | | CCC | | | CAC | | | CGC | | | C |
| | CUA | {close oversize brace} | Leu | CCA | {close oversize brace} | Pro | CAA | | | CGA | {close oversize brace} | Arg | A |
| | | | | | | | | {close oversize brace} | Gln | | | | |
| | CUG | | | CCG | | | CAG | | | CGG | | | G |
| A | AUU | | | ACU | | | AAU | | | AGU | | | U |
| | | | | | | | | {close oversize brace} | Asn | | {close oversize brace} | Ser | |
| | AUC | {close oversize brace} | Ile | ACC | | | AAC | | | AGC | | | C |
| | AUA | | | ACA | {close oversize brace} | Thr | AAA | | | AGA | | | A |
| | | | | | | | | {close oversize brace} | Lys | | {close oversize brace} | Arg | |
| | AUG | | Met | ACG | | | AAG | | | AGG | | | G |
| G | GUU | | | GCU | | | GAU | | | GGU | | | U |
| | | | | | | | | {close oversize brace} | Asp | | | | |
| | GUC | | | GCC | | | GAC | | | GGC | | | C |
| | GUA | {close oversize brace} | Val | GCA | {close oversize brace} | Ala | GAA | | | GGA | {close oversize brace} | Gly | A |
| | | | | | | | | {close oversize brace} | Glu | | | | |
| | GUG | | | GCG | | | GAG | | | GGG | | | G |

It should be understood that process steps within the following description of the method may be modified, changed and/or eliminated, depending on the particular preferences of the processor and/or the particular mechanical apparatus available to the processor, as well as the specific reagents and/or materials available and/or convenience and/or economics of use.

The glargine insulin analog prepared by the present invention may be formulated as liquid glargine insulin analog or crystalline glargine insulin analog. According to an embodiment of the invention, a preparation of recombinant liquid glargine insulin analog is in a substantially liquid form and that has not been through a crystallization process. Eliminating these steps has no negative impact on the purity of the liquid glargine insulin analog produced, but has the added advantage of reducing the amount of inactive insulin multimers in the liquid glargine insulin analog of the invention. Glargine insulin analog reconstituted from lyophilized and crystallized insulin may be contaminated with inactive insulin multimers and is less preferred.

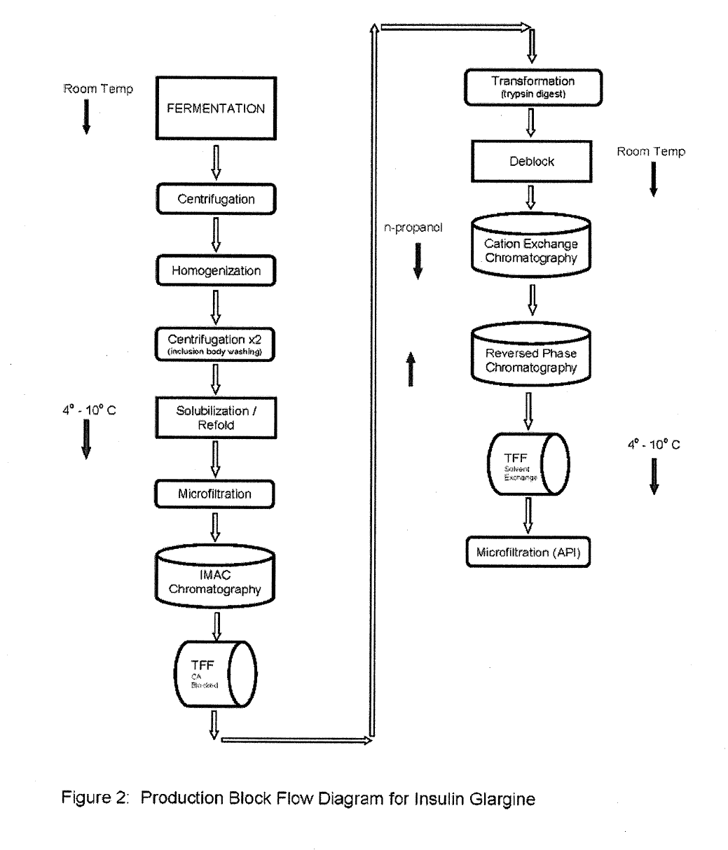

According to one embodiment, the methods of producing glargine insulin analog described herein generally include the following steps: fermentation/expression, Inclusion body isolation, solubilization of glargine proinsulin analog; refolding processing and transformation of glargine proinsulin analog to glargine insulin analog; and purification of glargine insulin analog. FIG. 2 illustrates a flow chart of preferred process steps in producing glargine insulin analog according to embodiments of the present invention.

Expression of glargine proinsulin analog may occur in a recombinant expression system. According to one embodiment, the recombinant expression system is a working cell bank (WCB) containing glargine proinsulin analog expressing vectors. For example, the cells of the WCB may be vertebrate or invertebrate cells, such as prokaryote or eukaryote cells, and most preferably the cells may be mammalian, bacterial, insect, or yeast cells. In one embodiment, the cell is a bacterial cell and in a further embodiment, the bacteria is E. coli. In another embodiment, the cell is a yeast cell and in a further embodiment, the yeast cell is S. cerevisiae or S. pombe.

In one embodiment, E. coli cells may be cultured and disrupted to provide a composition comprising inclusion bodies. The inclusion bodies contain the modified proinsulin sequence. The glargine proinsulin analogs expressed by cells of the WCB according to the method of the invention may be secreted from the cells and include a secretory sequence. In other embodiments, glargine proinsulin analogs expressed by cells of the WCB are not secreted from the cells, and thus do not include a secretory sequence.

The step of solubilizing the composition of inclusion bodies may involve adjusting the pH to achieve complete solubilization of the modified glargine proinsulin sequences. In one embodiment, the inclusion bodies may be solubilized by adjusting the pH to at least 10.5, preferably from 10.5 to 12.5, preferably from 11.8-12. The pH may be adjusted by adding an alkali hydroxide such as NaOH or KOH to the composition of inclusion bodies. In addition, the step of solubilization may use one or more reducing agents and/or chaotropic agent. Suitable reducing agents may include those selected from the group consisting of 2-mercaptoethanol, L-cysteine hydrochloride monohydrate, dithiothreitol, dithierythritol, and mixtures thereof. Suitable chaotropic agents include those selected from the group consisting of urea, thiourea, lithium perchlorate or guanidine hydrochloride, and mixtures thereof.

The solubilized inclusion bodies may be mixed in a refolding buffer, such as glycine or sodium carbonate, at a pH of 7-12, preferably from 10-11, preferably from 10.5-11, to refold the modified proinsulin sequences to a proinsulin derivative peptide, e.g., glargine proinsulin derivative peptide. The solution with refolded material should be pH adjusted to 7-9, preferably 7.8-8.2, with or without the addition of an alkaline salt, preferably sodium chloride to a final concentration of 100 mM to 1M final concentration, preferably 500 mM to 1M, preferably 700 mM, and may be filtered and loaded onto a column, such as an immobilized metal-ion affinity chromatography (IMAC) column Commercially available resins suitable for embodiments of the present invention include Nickle Sepharose 6 Fast Flow (GE Healthcare), Nickle NTA Agarose (GE Healthcare), Chelating Sepharose Fast flow(GE Healthcare), IMAC Fast Flow (GE Healthcare).

During the step of processing of glargine proinsulin to glargine insulin analog one or more of the amino acids may be protected to prevent side reactions and impurities during the cleavage step. In a further embodiment, the addition of a protecting group to glargine insulin analog may be added prior to addition of trypsin. In particular, protecting groups may be used to protect the lysine residue of the B-chain. A preferred protecting group is citriconic anhydride. In native human proinsulin, citriconic anhydride is preferably used to block Lys(B₂₉) in the proinsulin pro-lys-thr-arg-arg (SEQ ID NO: 24) amino acid sequence, and thus reducing the formation of desthreonine insulin impurity. In glargine proinsulin analog of the present invention, citriconic anhydride may also be used to block Lys(B₂₉) in the pro-lys-thr-arg-arg (SEQ ID NO: 24) amino acid sequence. The citriconic anhydride protecting group may reduce the formation of impurities such as desthreonine insulin and arg(B₃₁)-insulin.

In one embodiment, an excess molar ratio of citriconic anhydride to glargine proinsulin analog may be used. For example, about 10 fold molar excess or more of citriconic anhydride to glargine proinsulin analog may be suitable, and more preferably, about 20 fold molar excess or more. There is no upper limit on the excess molar ratio and the molar ratio may be as high as about 200 fold or about 300 fold.

After the citriconic anhydride blocking step, glargine proinsulin analog is subject to buffer exchange and concentration by tangential flow filtration or diafiltration. Proinsulin derivative peptide, with the blocking groups, may be enzymatically cleaved, preferred by subjecting the proinsulin derivative peptide to trypsin digestion. Although embodiments of the present invention may use commercially available rat, bovine, porcine or human trypsins or other isoenzymes or derivatives or variants thereof, it is also possible to use the following enzymes: trypsin from Fusarium oxysporum and from Streptomyces (S. griseus, S. exfoliatus, S. erythraeus, S. fradiae and S. albidoflavus), tryptase, mastin, acrosin, kallikrein, hepsin, prostasin I, lysyl endopeptidase (Lysin-C) and endoproteinase Arg-C (clostripain). In one embodiment, trypsin digestion occurs at pH from about 7 to 10, and more preferably from 8.8 to 9.2. In a further embodiment, the trypsin digest is quenched by adding glacial acetic acid. While it is contemplated that other additives may be employed, acetic acid appears to be most preferred and stable for this purpose.

Trypsin is an enzyme that has specific cleavage activity at the c-terminal side of arginine residues, and to a lesser extent, lysine residues, of the C-peptide. In the transformation reaction, it is required that the terminal arginine or lysine residues of the C-peptide be removed. In native human proinsulin, when trypsin cleaves at the lysine in position 64, it will be unable to remove the arginine at position 65, due to the fact that it requires at least one residue on both sides of a cleavage site. What results is the production of an unwanted by-product, arg(A₀)-insulin. This by-product constitutes a small loss in yield and generates an undesired contaminant. By converting this lysine 64 into another uncharged amino acid, particularly alanine, the arg(A₀)-insulin byproduct is preferentially not formed. When formed, less than 10%, and more preferably less than 0.3% of total byproducts from the trypsin transformation reaction may be arg(A₀). This is because the trypsin no longer acts to cleave at this particular site of the proinsulin derivative peptide.

The glargine insulin analog is subjected to deblocking after digestion with trypsin. Citriconic anhydride deblocking occurs by permitting the glargine insulin to be warmed to a temperature of 15° C. to 25° C., more preferably 18° C. to 20° C., and the pH is adjusted to 2.5 to 3.5, more preferably 2.8 to 3.0.

In one embodiment, after deblocking the glargine insulin is purified in a chromatography column, such as an ion exchange column Following the ion exchange chromatography, the glargine insulin may be further purified using reverse phase chromatography. In one embodiment, the intermediate solution may be purified in a chromatography column by eluting the glargine insulin analog using a buffer comprising an alcohol or organic solvent, n-propanol or acetonitrile. The buffer may also further comprise an alkali metal salt, preferable sodium sulfate. The buffer may also further comprise an organic acid, preferable phosphoric acid.

According to the invention, insulin having two additional arginine residues at the carboxyl terminal end of the B chain, along with glycine substituted for asparagines at the carboyl terminal end of the A chain allows glargine insulin to form a precipitate (hexamer) when injected subcutaneously. Accordingly, upon administration of this glargine insulin analogue to a patient, it can maintain a peakless level for up to 24 hours. In particular, this analogue is particularly suitable for moderate control of serum glucose levels that more closely resemble typical basal insulin secretion. For example, if administered prior to sleep, insulin glargine can reduce the risk of nocturnal hypoglycaemia.

According to one embodiment of the invention, the insulin glargine analogue is provided to a patient in combination with a rapid acting insulin to provide optimal glycemic control.

The manufacturing process described herein results in a preparation of glargine insulin analog in liquid active pharmaceutical ingredient (API) form.

According to one embodiment of the invention, the glargine insulin analog is provided to a patient in combination with human insulin or another insulin analog to provide optimal glycemic control.

In some embodiments, the preparations comprise a pharmaceutically acceptable preparation comprising recombinant glargine insulin analog and being essentially free of modified proinsulin sequences.

It should be understood that process steps within the following description of the method may be modified, changed and/or eliminated, depending on the particular preferences of the processor and/or the particular mechanical apparatus available to the processor, as well as the specific reagents and/or materials available and/or convenience and/or economics of use.

### EXAMPLE 1

### Preparation of an E. coli Clone Expressing Glargine Proinsulin

The preparation of transformed E. Coli containing cells capable of expressing recombinant glargine proinsulin is carried out according to the following processes. In addition, a cell bank of the transformed E. Coli is also described.

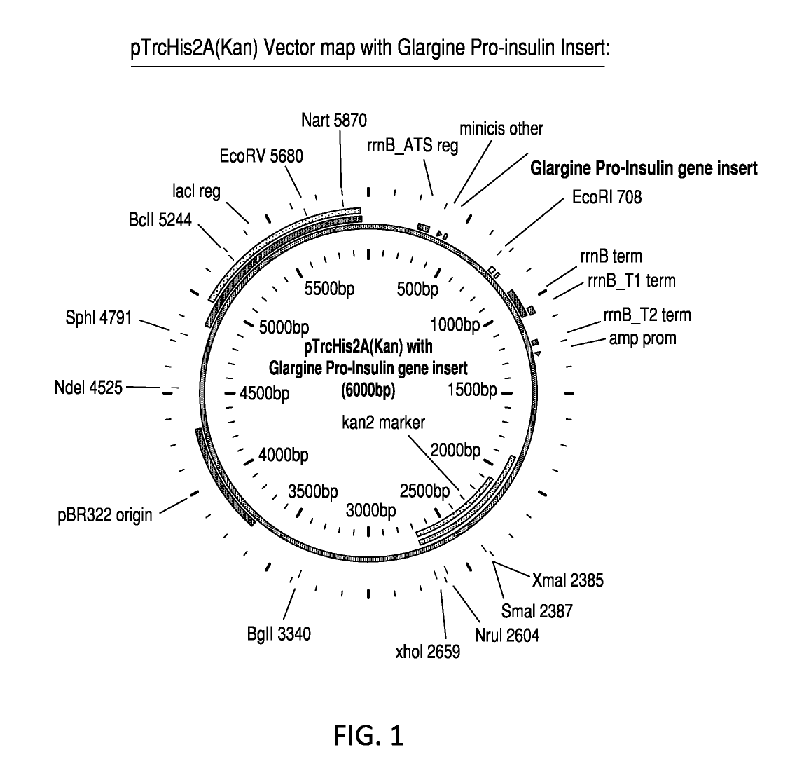

Step 1: Construction of a purified glargine proinsulin gene segment for insertion into the vector. The initial gene construct was synthesized in a basic cloning vector. The gene construct included the N-terminal histidine tag, MHHHHHHGGR (SEQ ID NO: 4), modified B-chain, and modified C-peptide with the alanine codon in place of the native lysine and having the amino acid sequence MHHHHHHGGRFVNQHLCGSHLVEALYLVCGERGFFYTPKTRREAEDLQVGQVELGGG PGAGSLQPLALEGSLQARGIVEQCCTSICSLYQLENYCG (SEQ ID NO: 16). The gene was flanked by Nde1 and EcoR1 restriction sites, for subsequent subcloning into the desired expression vector. The codons selected were optimized for expression in E. coli. The following sequence represents the pTrcHis2a(Kan) vector with glargine proinsulin insert (FIG. 1). The IPTG inducible promoter region which regulates the transcription rate is shown by the dotted underline, while the glargine proinsulin insert, adjacent the promoter region, is shown by the solid underlined. The sequence shown in bold and italics is the Kanamycin gene, which provides the antibiotic selection marker for the vector.

(SEQ ID NO: 25)

5′GTTTGACAGCTTATCATCGACTGCACGGTGCACCAATGCTTCTGGCGTCAGGCAGCCATCGG
AAGCTGTGGTATGGCTGTGCAGGTCGTAAATCACTGCATAATTCGTGTCGCTCAAGGCGCACTC

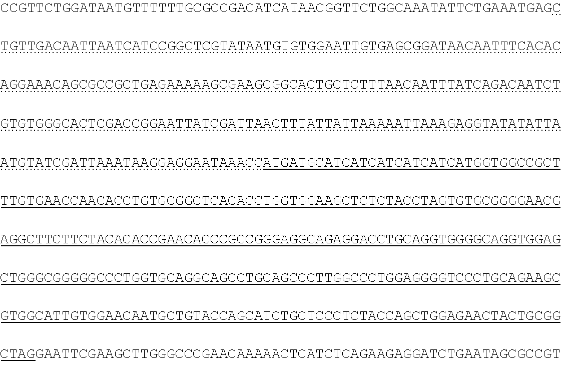

CGACCATCATCATCATCATCATTGAGTTTAAACGGTCTCCAGCTTGGCTGTTTTGGCGGATGAG
AGAAGATTTTCAGCCTGATACAGATTAAATCAGAACGCAGAAGCGGTCTGATAAAACAGAATTT
GCCTGGCGGCAGTAGCGCGGTGGTCCCACCTGACCCCATGCCGAACTCAGAAGTGAAACGCCGT
AGCGCCGATGGTAGTGTGGGGTCTCCCCATGCGAGAGTAGGGAACTGCCAGGCATCAAATAAAA
CGAAAGGCTCAGTCGAAAGACTGGGCCTTTCGTTTTATCTGTTGTTTGTCGGTGAACGCTCTCC
TGAGTAGGACAAATCCGCCGGGAGCGGATTTGAACGTTGCGAAGCAACGGCCCGGAGGGTGGCG
GGCAGGACGCCCGCCATAAACTGCCAGGCATCAAATTAAGCAGAAGGCCATCCTGACGGATGGC
CTTTTTGCGTTTCTACAAACTCTTTTTGTTTATTTTTCTAAATACATTCAAATATGTATCCGCT
CATGAGACAATAACCCTGATAAATGCTTCAATAATATTGAAAAAGGAAGAGTATGAGTATTCAA
CATTTCCGTGTCGCCCTTATTCCCTTTTTTGCGGCATTTTGCCTTCCTGTTTTTGCTCACCCAG
AAACGCTGGTGAAAGTAAAAGATGCTGAAGATCAGTTGGGTGCACGAGTGGGTTACATCGAACT
GGATCTCAACAGCGGTAAGATCCTTGAGAGTTTTCGCCCCGAAGAACGTTTTCCAATGATGAGC
ACTTTTAAAGTTCTGCTATGTGGCGCGGTATTATCCCGTGTTGACGCCGGGCAAGAGCAACTCG
GTCGCCGCATACACTATTCTCAGAATGACTTGGTTGAGTCCTGAATCGCCCCATCATCCAGCCA
GAAAGTGAGGGAGCCACGGTTGATGAGAGCTTTGTTGTAGGTGGACCAGTTGGTGATTTTGAAC
TTTTGCTTTGCCACGGAACGGTCTGCGTTGTCGGGAAGATGCGTGATCTGATCCTTCAACTCAG
CAAAAGTTCGATTTATTCAACAAAGCCGCCGTCCCGTCAAGTCAGCGTAATGCTCTGCCAGTGT

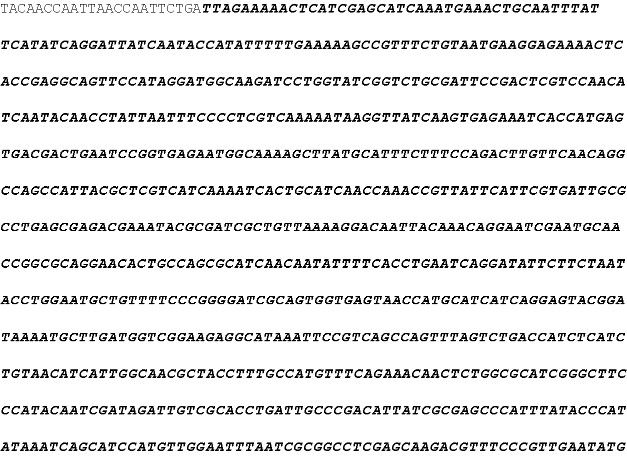

GCTCATAACACCCCTTGTATTACTGTTTATGTAAGCAGACAGTTTTATTGTTCATGATGATATA
TTTTTATCTTGTGCAATGTAACATCAGAGATTTTGAGACACAACGTGGCTTTGTTGAATAAATC
GAACTTTTGCTGAGTTGAAGGATCAGATCACGCATCTTCCCGACAACGCAGACCGTTCCGTGGC
AAAGCAAAAGTTCAAAATCACCAACTGGTCCACCTACAACAAAGCTCTCATCAACCGTGGCTCC
CTCACTTTCTGGCTGGATGATGGGGCGATTCAGGACTCACCAGTCACAGAAAAGCATCTTACGG
ATGGCATGACAGTAAGAGAATTATGCAGTGCTGCCATAACCATGAGTGATAACACTGCGGCCAA
CTTACTTCTGACAACGATCGGAGGACCGAAGGAGCTAACCGCTTTTTTGCACAACATGGGGGAT
CATGTAACTCGCCTTGATCGTTGGGAACCGGAGCTGAATGAAGCCATACCAAACGACGAGCGTG
ACACCACGATGCCTGTAGCAATGGCAACAACGTTGCGCAAACTATTAACTGGCGAACTACTTAC
TCTAGCTTCCCGGCAACAATTAATAGACTGGATGGAGGCGGATAAAGTTGCAGGACCACTTCTG
CGCTCGGCCCTTCCGGCTGGCTGGTTTATTGCTGATAAATCTGGAGCCGGTGAGCGTGGGTCTC
GCGGTATCATTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTGCCTCACTGATT
AAGCATTGGTAACTGTCAGACCAAGTTTACTCATATATACTTTAGATTGATTTAAAACTTCATT
TTTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATACC
AAATACTGTCCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATAGTCCTGTCGG
GTTTCGCCACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCTCACATGT
TCTTTCCTGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAAGAGCGCCTG
ATGCGGTATTTTCTCCTTACGCATCTGTGCGGTATTTCACACCGCATATGGTGCACTCTCAGTA
CAATCTGCTCTGATGCCGCATAGTTAAGCCAGTATACACTCCGCTATCGCTACGTGACTGGGTC
ATGGCTGCGCCCCGACACCCGCCAACACCCGCTGACGCGCCCTGACGGGCTTGTCTGCTCCCGG
CATCCGCTTACAGACAAGCTGTGACCGTCTCCGGGAGCTGCATGTGTCAGAGGTTTTCACCGTC
ATCACCGAAACGCGCGAGGCAGCAGATCAATTCGCGCGCGAAGGCGAAGCGGCATGCATTTACG
TTGACACCATCGAATGGTGCAAAACCTTTCGCGGTATGGCATGATAGCGCCCGGAAGAGAGTCA
ATTCAGGGTGGTGAATGTGAAACCAGTAACGTTATACGATGTCGCAGAGTATGCCGGTGTCTCT
TATCAGACCGTTTCCCGCGTGGTGAACCAGGCCAGCCACGTTTCTGCGAAAACGCGGGAAAAAG
TGGAAGCGGCGATGGCGGAGCTGAATTACATTCCCAACCGCGTGGCACAACAACTGGCGGGCAA
ACAGTCGTTGCTGATTGGCGTTGCCACCTCCAGTCTGGCCCTGCACGCGCCGTCGCAAATTGTC
GCGGCGATTAAATCTCGCGCCGATCAACTGGGTGCCAGCGTGGTGGTGTCGATGGTAGAACGAA
GCGGCGTCGAAGCCTGTAAAGCGGCGGTGCACAATCTTCTCGCGCAACGCGTCAGTGGGCTGAT
CATTAACTATCCGCTGGATGACCAGGATGCCATTGCTGTGGAAGCTGCCTGCACTAATGTTCCG
GCGTTATTTCTTGATGTCTCTGACCAGACACCCATCAACAGTATTATTTTCTCCCATGAAGACG
GTACGCGACTGGGCGTGGAGCATCTGGTCGCATTGGGTCACCAGCAAATCGCGCTGTTAGCGGG
CCCATTAAGTTCTGTCTCGGCGCGTCTGCGTCTGGCTGGCTGGCATAAATATCTCACTCGCAAT
CAAATTCAGCCGATAGCGGAACGGGAAGGCGACTGGAGTGCCATGTCCGGTTTTCAACAAACCA
TGCAAATGCTGAATGAGGGCATCGTTCCCACTGCGATGCTGGTTGCCAACGATCAGATGGCGCT
GGGCGCAATGCGCGCCATTACCGAGTCCGGGCTGCGCGTTGGTGCGGATATCTCGGTAGTGGGA
TACGACGATACCGAAGACAGCTCATGTTATATCCCGCCGTCAACCACCATCAAACAGGATTTTC
GCCTGCTGGGGCAAACCAGCGTGGACCGCTTGCTGCAACTCTCTCAGGGCCAGGCGGTGAAGGG
CAATCAGCTGTTGCCCGTCTCACTGGTGAAAAGAAAAACCACCCTGGCGCCCAATACGCAAACC
GCCTCTCCCCGCGCGTTGGCCGATTCATTAATGCAGCTGGCACGACAGGTTTCCCGACTGGAAA
GCGGGCAGTGAGCGCAACGCAATTAATGTGAGTTAGCGCGAATTGATCTG

The modified proinsulin sequence without the tag is as follows:

(SEQ ID NO: 26)
TTTGTGAACCAACACCTGTGCGGCTCACACCTGGTGGAAGCTCTCTACCT
AGTGTGCGGGGAACGAGGCTTCTTCTACACACCGAACACCCGCCGGGAGG
CAGAGGACCTGCAGGTGGGGCAGGTGGAGCTGGGCGGGGGCCCTGGTGCA
GGCAGCCTGCAGCCCTTGGCCCTGGAGGGGTCCCTGCAGAAGCGTGGCAT
TGTGGAACAATGCTGTACCAGCATCTGCTCCCTCTACCAGCTGGAGAACT
ACTGCGGCTAG

Step 2: Generation of the pTrcHis2A(Kan) vector containing glargine proinsulin. Commercially available pTrcHis2A(Kan) vector was modified to include a Kanamycin resistance gene in the middle of the Ampicillin resistance gene to negate the Ampicillin resistance prior to insertion of the proinsulin sequence into the vector. Ampicillin resistance heightens the potential for allergic reactions to preparations made using vector constructs that include the Ampicillin resistance gene. Therefore it is preferable to eliminate the Ampicillin resistance in the constructs that are prepared and used.

The pTrcHis2A(Kan) vector was modified at the start codon in the multiple cloning site by replacing the Ncol restriction site with an Ndel site to simplify subsequent subcloning work.
Nco1=CCATGG-Nde1=CATATG

The proinsulin gene was isolated from the DNA 2.0 plasmid using Nde1 to cleave at the N-terminal side of the gene and EcoR1 to cleave at the C-terminal side of the gene. The Digested DNA was run over a 2% agarose gel to separate the plasmid DNA from the glargine proinsulin gene. A QIAquick™ (Qiagen) gel purification kit was then used to purify the gene construct.

Accordingly, a sequential digest of the vector with Nde1 and EcoR1, respectively, was performed. The vector DNA was also purified using a QIAquick gel purification kit. Following purification of the vector and the gene, a 5′ Nde1 and a 3′ EcoR1 ligation reaction were utilized to insert the proinsulin gene into the pTrcHis2A(Kan) vector.

Step 3: Transformation. One microliter of the ligation reaction was used to transform competent E. coli cells BL21 with the pTrcHis2A(Kan) plasmid containing the proinsulin gene. The transformed E. coli BL21 cells were plated on LB-Kan agar plates and incubated overnight at 37° C. Several clones were selected and sequenced. Clones with the correct sequence were then screened for expression.

The resulting clone is referred to as the glargine proinsulin pTrcHis2A(Kan) vector.

Step 4: Preparation of the working cell bank (WCB). To establish the WCB, sterile growth medium was inoculated with the recombinant BL21 E. coli containing the glargine proinsulin Met-His-tagged/pTrcHis2A(Kan) vector and incubated to allow cell growth. The cells were harvested in an ISO5 (class 100) environment under a biosafety cabinet and then sterile filtered. Sterile medium and glycerol were added to the sterile filtered cells. 1 mL aliquots of the cells were then dispensed into sterile ampoules and stored at -80° C. Aseptic techniques were utilized to generate the WCB.

### EXAMPLE 2

### Product Manufacture of Glargine Insulin Analog from Modified Proinsulin Sequence

Step 1—Culturing of E. coli transformed with glargine modified proinsulin sequence from the WCB of Example 1. Seed an inoculum preparation of the WCB in a sterile growth medium that includes yeastolate (purchased from VWR, Prod. #90004-426 or -488), select phytone, sodium chloride, purified water, sterile Kanamycin solution), and incubate until growth to an Optical density (OD_{600 nm}) of 2 to 4. Prepare a fermentation media (containing select phytone, yeastolate, glycerin, BioSpumex 153K (Cognis, Inc.) in a fermentor. Add the following sterilized phosphate solutions to the Fermentor. Prepare a Phosphate flask 1—potassium phosphate monobasic and potassium phosphate dibasic containing Kanamycin solution. Prepare a Phosphate flask 2—potassium phosphate monobasic and potassium phosphate dibasic. Add seed inoculate of E. coli to the Fermentor—growth to O.D. (optical density) 600 nm of 8 to 10 (mid log phase). Add a dioxane free IPTG (purchased from Promega, Catalog No. #PA V3953 (VWR Catalog #PAV3953) solution to the fermentor (to induce transcription of the K64A glargine proinsulin gene). Incubate for 4 hours. This results in the production of a concentrated cell suspension containing His-tagged glargine proinsulin inclusion bodies. The cell suspension is then centrifuged to provide a cell paste for the subsequent inclusion body isolation step.

Step 2—Disruption—Cells containing inclusion bodies expressing glargine modified proinsulin sequence are lysed in a basic Tris/salt buffer, using a Niro Soavi homogenizer (1100-1200 bar).

Step 3—Inclusion Body Washing—Contaminant protein removal is accomplished via two sequential washes with a Tris/Triton X-100 buffer, followed by two sequential washes with a Tris/Tween-20 buffer, and finally a single wash with a Tris/NaCl buffer.

Step 4—Solubilization—Inclusion bodies enriched with the modified glargine proinsulin peptide are solubilized in 4-8M urea, preferably 6-8M urea, containing reducing agents (2-mercaptoethanol, L-cysteine hydrochloride monohydrate). Complete solubilization is achieved by adjusting the pH to 10.5-12, preferably 11.8-12 with NaOH.

Step 5—Dilution refolding—The solubilized glargine insulin analog protein is then diluted into refolding buffer (20 mM Glycine, pH 10-11 at 6-10° C.) to a final concentration of 1.5 mg/ml and permitted to refold for 24 to 72 hours, preferentially about 48 hours, at 6-10° C. Higher protein concentration may be used in the refold if desired, however, overall refold efficiency will decrease. Sodium Chloride and Phosphate are then added to final concentrations of 700 mM and 25 mM respectively, followed by pH adjustment to 7.0 to 9.0, preferably 7.9-8.0 with 6M HCl.

Step 6—IMAC Chromatography—The dilute proinsulin derivative is loaded onto an IMAC column to a maximum capacity of ≦26.5 mg main peak protein per ml of resin. A 75 mM imidizole buffer is used to isocratically strip the majority of impurities from the column Tagged glargine proinsulin is then eluted isocratically using ≦300 mM imidizole.

Step 7—Citriconic anhydride(CA) Blocking—To the IMAC pool, add citriconic anhydride at a molar ratio of 20:1 (CA to Glargine tagged proinsulin), while stirring at 4-10° C. Allow the sample to stir for not less than 3 hour at 4-10° C.

Step 8—Buffer exchange—To the blocked material, add EDTA to a final concentration of 20 mM. Exchange the buffer using a membrane with a suitable molecular weight cutoff (e.g. 3000 Da). The final buffer should be at least 97% exchanged to a 20 mM Tris-Cl, pH 7.0-10.0, preferably 8.8 to 9.2 at 8-10° C. A protein concentration of approximately 5 mg/ml is desirable.

Step 9—Trypsin Enzymatic Transformation/Proteolysis—The buffer exchanged sample is digested with a 1000:1 mass ratio of main peak protein to trypsin, in the presence of 5 mM CaCl. The ratio of trypsin may be increased or decreased depending on the desired length of time for the reaction. Once complete, based on HPLC, the digest is then quenched by the addition of acetic acid to ≧700 mM.

Step 10—Citriconic anhydride Deblocking—The trypsin digest solution is then warmed to 18 to 20° C. and the pH is adjusted to 2.8 to 3.0. The digest is stored at room temperature for not less than 10 hours to permit release of the citriconic anhydride.

### EXAMPLE 3

### Final Purification

Step 11—Ion Exchange Chromatography—The digested material is loaded onto a cation exchange column and eluted with a NaCl gradient, in the presence of 20% n-propanol or acetonitrile at pH 2-5, preferably 4.0. RP-HPLC is used to pool the appropriate fractions containing the Glargine insulin peak of interest at the desired purity level.

Step 12—Reverse Phase Chromatography—The S-column pool containing the Glargine insulin is loaded onto an RPC30 or C18 reverse phase column and eluted using an n-propanol or acetonitrile gradient in the presence of 200 mM sodium sulfate and 0.136% phosphoric acid. Fractions are immediately diluted 1:4 with water if n-propanol is used for elution; or 1:2 with water if acetonitrile is used for elution, or no dilution if acetonitrile is used for elution. RP-HPLC is used to pool the appropriate fractions containing the glargine insulin peak of interest at the desired purity level.

Step 13—Buffer Exchange—Exchange the sample into WFI (water for injection) using a membrane with a suitable molecular weight cutoff (e.g. 3000 Da). The pH of the solution should be monitored and maintained at 2-5, preferably 3.5 to 4.0. The final sample is concentrated to 5-8 mg/ml, with an adjusted pH of 2-5, preferably 3.8 to 4.2, chilled to 6-10° C. This material represents the liquid API form of the presently disclosed preparations of Glargine Insulin Analog. The API should be stored in the dark at 6-10° C.

### EXAMPLE 4

### API Formulation

The glargine Insulin Analog purified by Example 3 is formulated by diluting the API material with cold WFI to a final concentration of 4.54725 mg/ml. A concentrated formulation buffer stock containing 85 mg/ml glycerol, 13.5 mg/ml meta cresol, 0.150 mg/ml zinc chloride and polysorbate(20) 0.1 mg/ml is added to the API material in a 1/5 ratio of formulation buffer stock to API. The solution is mixed, followed by sterile filtration into appropriate vials in 10 ml aliquots.

### EXAMPLE 5

### Working Cell Bank

The preparation of a WCB (working cell bank) for research and development containing cells capable of expressing recombinant glargine proinsulin is carried out according to the following processes:

The cloning procedure outlined in Example 1 is utilized to create the initial vector. Purified His Tagged Glargine proinsulin pTrcHis2A(Kan) vector is transformed into competent BL21 E. coli cells and plated on sterile LB-Kan plates. From the plates, an isolated colony is used to inoculate sterile LB-Kan media (˜100 mls) The cells are grown at 37° C. to mid log phase (˜4-5 hours) OD_{600 nm} of ˜1.5-2.0. Culture media containing cells is then aliquoted into sterile cryovials, combined with glycerol at a 20% final concentration. The vials are then stored at −80° C.

### EXAMPLE 6

### Comparative Analysis

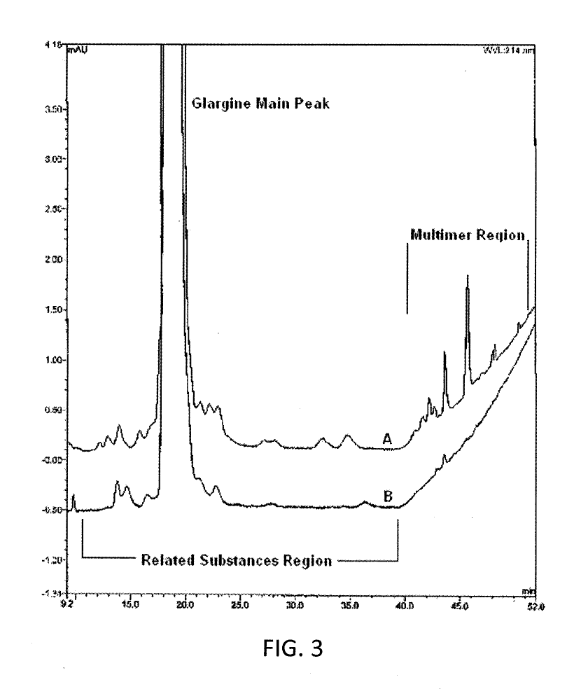

The present example demonstrates the enhanced purity of the glargine analog product according to Example 4. FIG. 3 depicts an analytical HPLC overlay of LANTUS® (A) and the glargine analog (B). The glargine analog demonstrates increased purity with respect to related substances and multimeric species over LANTUS®. In the related substance region, the glarine analog shows noticeably lower levels of contaminants in both the related substance region and the multimeric region. Most notably the number of multimeric species is much lower in the glargine analog. Overall purity for the LANTUS® material (A) in the current profile was 98.8%, while the glargine product produced by the herein described method (B) was 99.6%.

What is claimed is:

1. A composition comprising a proinsulin sequence having the formula
R₁—(B₁-B₂₉)—B₃₀—R₂—R₃—X—R₄—R₅-(A₁-A₂₀)-A₂₁-R₆ Formula I
wherein
R₁ is a tag sequence containing one or more amino acids or R₁ is absent with an Arg or Lys present prior to the start of the B chain;
(B₁-B₂₉) and (A₁-A₂₀) comprise amino acid sequences of native human insulin;
B₃₀ is Gly, Ala, Ser, Thr, Val, Leu, Ile, Asn, Gln, Cys, Met, Tyr, Phe, Pro, or Trp;
R₂, R₃ and R₅ are Arg;
R₄ is any amino acid other than Gly, Lys or Arg or is absent;
X is a sequence comprises one or more amino acids or is absent, provided that X does not comprise a C-terminal Gly, Lys, or Arg when R₄ is absent;
A₂₁ is Gly, Ala, Val, Leu, Ile, Pro, Phe, Trp, Met, Ser, Thr, Tyr, Asp, or Glu; and
R₆ is a tag sequence containing one or more amino acids or R₆ is absent.

2. The composition of claim 1, wherein R₁ and/or R₆ is present and R₁ is tag sequence of one or more amino acids with a C-terminal Arg or Lys and/or R₆ tag sequence of one or more amino acids with a N-terminal Arg or Lys.

3. The composition of claim 1, wherein R₄ is Ala.

4. The composition of claim 1, wherein the modified proinsulin sequence comprises a connecting peptide sequence of a sequence having the formula
R₂—R₃—X—R₄—R₅ Formula II
wherein
R₂, R₃, R₄, R₅, and X are defined in claim 1.

5. The composition of claim 4, wherein the connecting peptide sequence is
(SEQ ID NO: 8)
RREAEDLQVGQVELGGGPGAGSLQPLALEGSLQAR.

6. The composition of claim 1, wherein the modified proinsulin sequence is
(SEQ ID NO: 15)
FVNQHLCGSHLVEALYLVCGERGFFYTPKTRREAEDLQVGQVELGGGPG
AGSLQPLALEGSLQARGIVEQCCTSICSLYQLENYCG.

7. The composition of claim 1, wherein the modified proinsulin sequence is
(SEQ ID NO: 17)
MALWMRLLPLLALLALWGPDPAAAFVNQHLCGSHLVEALYLVCGERGF
FYTPKTRREAEDLQVGQVELGGGPGAGSLQPLALEGSLQARGIVEQCC
TSICSLYQLENYCG.

8. The composition of claim 1, wherein the modified proinsulin sequence is
(SEQ ID NO: 16)
MHHHHHHGGRFVNQHLCGSHLVEALYLVCGERGFFYTPKTRREAEDLQ
VGQVELGGGPGAGSLQPLALEGSLQARGIVEQCCTSICSLYQLENYCG.

9. The composition of claim 1, wherein the modified proinsulin sequence is
(SEQ ID NO: 18)
MHHHHHHGGRFVNQHLCGSHLVEALYLVCGERGFFYTPKTRREAEDLQ
VGQVELGGGPGAGSLQPLALEGSLQARGIVEQCCTSICSLYQLENYCG
RHHHHHH.

10. The composition of claim 1, wherein the modified proinsulin sequence is
(SEQ ID NO: 19)
MHHHHHHGGRFVNQHLCGSHLVEALYLVCGERGFFYTPKTRREAEDLQ
VGQVELGGGPGAGSLQPLALEGSLQARGIVEQCCTSICSLYQLENYCG
KHHHHHH.

11. The composition of claim 1, wherein the modified proinsulin sequence is
(SEQ ID NO: 20)
MRFVNQHLCGSHLVEALYLVCGERGFFYTPKTRREAEDLQVGQVELG
GGPGAGSLQPLALEGSLQARGIVEQCCTSICSLYQLENYCGRHHHHH
H.

12. The composition of claim 1, wherein the modified proinsulin sequence is
(SEQ ID NO: 21)
MRFVNQHLCGSHLVEALYLVCGERGFFYTPKTRREAEDLQVGQVELG
GGPGAGSLQPLALEGSLQARGIVEQCCTSICSLYQLENYCGKHHHHHH.

13. An expression vector comprising the nucleic acid sequence of claim 1.

14. The expression vector of claim 13, wherein the expression vector is His Tagged Glargine proinsulin pTrcHis2A(Kan).

15. A microorganism transformed with the vector of claim 14.

16. The microorganism of claim 15, further defined as an E. coli transformed with plasmid His Tagged Glargine proinsulin pTrcHis2A(Kan).

17. A process for producing glargine insulin analogs comprising the steps of:
(a) culturing E. coli cells under conditions suitable for expression of a modified proinsulin sequence having the formula
R₁—(B₁-B₂₉)—B₃₀—R₂—R₃—X—R₄—R₅-(A₁-A₂₀)-A₂₁-R₆ Formula I
wherein
R₁ is a tag sequence containing one or more amino acids or R₁ is absent with an Arg or Lys present prior to the start of the B chain;
(B₁-B₂₉) and (A₁-A₂₀) comprise amino acid sequences of native human insulin;
B₃₀ is Gly, Ala, Ser, Thr, Val, Leu, Ile, Asn, Gln, Cys, Met, Tyr, Phe, Pro, or Trp;
R₂, R₃ and R₅ are Arg;
R₄ is any amino acid other than Gly, Lys or Arg or is absent;
X is a sequence comprises one or more amino acids or is absent, provided that X does not comprise a C-terminal Gly, Lys, or Arg when R₄ is absent;
A₂₁ is Gly, Ala, Val, Leu, Ile, Pro, Phe, Trp, Met, Ser, Thr, Tyr, Asp, or Glu; and
R₆ is a tag sequence containing one or more amino acids or R₆ is absent;
(b) disrupting said cultured E. coli cells to provide a composition comprising inclusion bodies containing the modified proinsulin sequence;
(c) solubilizing said composition of inclusion bodies; and
(d) recovering the glargine insulin analogs from said solubilized composition.

18. The process of claim 17, wherein the step of recovering the glargine insulin analogs further comprises:
(e) folding said modified proinsulin sequence to provide a proinsulin derivative peptide;
(f) purifying said proinsulin derivative peptide in a metal affinity chromatography column;
(g) protecting a Lys amino acid residue of the proinsulin derivative peptide with one or more protecting compounds;
(h) enzymatically cleaving said blocked proinsulin derivative peptide to remove a connecting peptide and provide an intermediate solution comprising glargine insulin analog; and
(i) purifying said intermediate solution using chromatography column(s) to yield the glargine insulin analog.

19. The process of claim 18, wherein the one or more protecting compounds comprise citriconic anhydride.

20. The process of claim 17, wherein the solubilization of said composition of inclusion bodies use one or more chaotropic agents selected from the group consisting of urea, thiourea, lithium perchlorate or guanidine hydrochloride and mixtures thereof.

21. A process for producing glargine insulin analogs comprising the steps of:
(a) providing a modified proinsulin sequence having the formula
R₁—(B₁-B₂₉)—B₃₀—R₂—R₃—X—R₄—R₅-(A₁-A₂₀)-A₂₁-R₆ Formula I
wherein
R₁ is a tag sequence containing one or more amino acids or R₁ is absent with an Arg or Lys present prior to the start of the B chain;
(B₁-B₂₉) and (A₁-A₂₀) comprise amino acid sequences of native human insulin;
B₃₀ is Gly, Ala, Ser, Thr, Val, Leu, Ile, Asn, Gln, Cys, Met, Tyr, Phe, Pro, or Trp;
R₂, R₃ and R₅ are Arg;
R₄ is any amino acid other than Gly, Lys or Arg or is absent;
X is a sequence comprises one or more amino acids or is absent, provided that X does not comprise a C-terminal Gly, Lys, or Arg when R₄ is absent;
A₂₁ is Gly, Ala, Val, Leu, Ile, Pro, Phe, Trp, Met, Ser, Thr, Tyr, Asp, or Glu; and
R₆ is a tag sequence containing one or more amino acids or R₆ is absent;
(b) folding said modified proinsulin sequence to provide a proinsulin derivative peptide;
(c) purifying said proinsulin derivative peptide in a metal affinity chromatography column;
(d) enzymatically cleaving said proinsulin derivative peptide to remove a connecting peptide and provide an intermediate solution comprising glargine insulin analog; and
(e) purifying said intermediate solution using chromatography column(s) to yield the glargine insulin analog.

22. The process of claim 21, further comprising
(f) protecting a lys amino acid residue of the proinsulin derivative peptide with one or more protecting compounds, prior to enzymatic cleavage.

23. The process of claim 22, wherein the one or more protecting compounds comprise citriconic anhydride.


SEQUENCE LISTING (26 sequences)

SEQ ID NO: 1 - protein, 5 aa, Artificial Sequence
  Description of Artificial Sequence Synthetic peptide
YPGDV

SEQ ID NO: 2 - protein, 24 aa, Unknown
  Description of Unknown Native pre-peptide
MALWMRLLPLLALLALWGPDPAAA

SEQ ID NO: 3 - protein, 6 aa, Artificial Sequence
  Description of Artificial Sequence Synthetic 6xHis tag
HHHHHH

SEQ ID NO: 4 - protein, 10 aa, Artificial Sequence
  Description of Artificial Sequence Synthetic peptide
MHHHHHHGGR

SEQ ID NO: 5 - protein, 21 aa, Unknown
  Description of Unknown Native insulin A-chain peptide
GIVEQCCTSICSLYQLENYCN

SEQ ID NO: 6 - protein, 30 aa, Unknown
  Description of Unknown Native insulin B-chain peptide
FVNQHLCGSHLVEALYLVCGERGFFYTPKT

SEQ ID NO: 7 - protein, 31 aa, Artificial Sequence
  Description of Artificial Sequence Synthetic polypeptide
EAEALQVGQVELGGGPGAGSLQPLALEGSLQ

SEQ ID NO: 8 - protein, 35 aa, Artificial Sequence
  Description of Artificial Sequence Synthetic polypeptide
RREAEDLQVGQVELGGGPGAGSLQPLALEGSLQAR

SEQ ID NO: 9 - protein, 35 aa, Artificial Sequence
  Description of Artificial Sequence Synthetic polypeptide
RREAEDLQVGQVGLGGGPGAGSLQPLALEGSLQAR

SEQ ID NO: 10 - protein, 35 aa, Artificial Sequence
  Description of Artificial Sequence Synthetic polypeptide
RREAEALQVGQVGLGGGPGAGSLQPLALEGSLQAR

SEQ ID NO: 11 - protein, 35 aa, Artificial Sequence
  Description of Artificial Sequence Synthetic polypeptide
RREAEDLQVGQVELGGGPGAGSLQPLAIEGSLQAR

SEQ ID NO: 12 - protein, 35 aa, Artificial Sequence
  Description of Artificial Sequence Synthetic polypeptide
RREAEDLQVGQVGLGGGPGAGSLQPLAIEGSLQAR

SEQ ID NO: 13 - protein, 35 aa, Artificial Sequence
  Description of Artificial Sequence Synthetic polypeptide
RREAEALQVGQVGLGGGPGAGSLQPLAIEGSLQAR

SEQ ID NO: 14 - protein, 35 aa, Artificial Sequence
  Description of Artificial Sequence Synthetic polypeptide
RREAEALQVGQVELGGGPGAGSLQPLALEGSLQAR

SEQ ID NO: 15 - protein, 86 aa, Artificial Sequence
  Description of Artificial Sequence Synthetic polypeptide
FVNQHLCGSHLVEALYLVCGERGFFYTPKTRREAEDLQVGQVELGGGPGAGSLQPLALEGSLQARGIVEQCCTSICSLYQLENYCG

SEQ ID NO: 16 - protein, 96 aa, Artificial Sequence
  Description of Artificial Sequence Synthetic polypeptide
MHHHHHHGGRFVNQHLCGSHLVEALYLVCGERGFFYTPKTRREAEDLQVGQVELGGGPGAGSLQPLALEGSLQARGIVEQCCTSICSLYQLENYCG

SEQ ID NO: 17 - protein, 110 aa, Artificial Sequence
  Description of Artificial Sequence Synthetic polypeptide
MALWMRLLPLLALLALWGPDPAAAFVNQHLCGSHLVEALYLVCGERGFFYTPKTRREAEDLQVGQVELGGGPGAGSLQPLALEGSLQARGIVEQCCTSICSLYQLENYCG

SEQ ID NO: 18 - protein, 103 aa, Artificial Sequence
  Description of Artificial Sequence Synthetic polypeptide
MHHHHHHGGRFVNQHLCGSHLVEALYLVCGERGFFYTPKTRREAEDLQVGQVELGGGPGAGSLQPLALEGSLQARGIVEQCCTSICSLYQLENYCGRHHHHHH

SEQ ID NO: 19 - protein, 103 aa, Artificial Sequence [sequence omitted]

SEQ ID NO: 20 - protein, 95 aa, Artificial Sequence
  Description of Artificial Sequence Synthetic polypeptide
MRFVNQHLCGSHLVEALYLVCGERGFFYTPKTRREAEDLQVGQVELGGGPGAGSLQPLALEGSLQARGIVEQCCTSICSLYQLENYCGRHHHHHH

SEQ ID NO: 21 - protein, 95 aa, Artificial Sequence
  Description of Artificial Sequence Synthetic polypeptide
MRFVNQHLCGSHLVEALYLVCGERGFFYTPKTRREAEDLQVGQVELGGGPGAGSLQPLALEGSLQARGIVEQCCTSICSLYQLENYCGKHHHHHH

SEQ ID NO: 22 - protein, 21 aa, Artificial Sequence
  Description of Artificial Sequence Synthetic peptide
GIVEQCCTSICSLYQLENYCG

SEQ ID NO: 23 - protein, 32 aa, Artificial Sequence
  Description of Artificial Sequence Synthetic polypeptide
FVNQHLCGSHLVEALYLVCGERGFFYTPKTRR

SEQ ID NO: 24 - protein, 5 aa, Artificial Sequence
  Description of Artificial Sequence Synthetic peptide
PKTRR

SEQ ID NO: 25 - DNA, 6000 bp, Artificial Sequence
  Description of Artificial Sequence Synthetic polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgatgcatcatcatcatcatcatggtggccgctttgtgaaccaacacctgtgcggctcacacctggtggaagctctctacctagtgtgcggggaacgaggcttcttctacacaccgaacacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcggctaggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 26 - DNA, 261 bp, Artificial Sequence
  Description of Artificial Sequence Synthetic polynucleotide
tttgtgaaccaacacctgtgcggctcacacctggtggaagctctctacctagtgtgcggggaacgaggcttcttctacacaccgaacacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcggctag